(19) United States
(12) Patent Application Publication
(10) Pub. No.: US 2014/0257362 A1
(43) Pub. Date: Sep. 11, 2014

(54) FILTERING AND REMOVING PARTICULATES FROM BLOODSTREAM

(71) Applicant: St. Jude Medical, Cardiology Division, Inc., St. Paul, MN (US)

(72) Inventor: Tracee Eidenschink, Wayzata, MN (US)

(73) Assignee: ST. JUDE MEDICAL, CARDIOLOGY DIVISION, INC., St. Paul, MN (US)

(21) Appl. No.: 14/178,780

(22) Filed: Feb. 12, 2014

Related U.S. Application Data
(60) Provisional application No. 61/773,893, filed on Mar. 7, 2013.

(51) Int. Cl. A61F 2/01

(52) U.S. Cl. CPC A61F 2/013; A61F 2002/011
USPC 606/200

(57) ABSTRACT

A system and method to protect blood vessels from blockage by various debris. A vessel protector of the system may include a body formed from a filtering material, the body having a collapsed configuration and a tubular expanded configuration with an open proximal end and an open distal end, a first snare attached to one end of the body, and a first pull-wire connected to the first snare, whereby the exertion of a pulling force on the first pull-wire contracts the first snare and thereby closes the one end of the body.

### CROSS-REFERENCE TO RELATED APPLICATIONS

The present application claims the benefit of the filing date of U.S. Provisional Patent Application No. 61/773,893 filed Mar. 7, 2013, the disclosure of which is hereby incorporated herein by reference.

### BACKGROUND OF THE INVENTION

The present invention is related to protecting against embolism, and more particularly to devices, systems, and methods for the filtration and removal of debris within blood vessels.

A frequent risk in medical procedures is the risk that the procedure will give rise to the formation of potentially life-threatening debris in the patient's bloodstream. Such debris may take the form of plaque or thrombi, which may travel through the patient's vasculature and become lodged in a position that blocks blood flow. For example, during coronary interventions, plaque may become dislodged and/or thrombi may form, both of which could migrate to the carotid arteries, possibly blocking the carotid arteries and causing a stroke.

### BRIEF SUMMARY OF THE INVENTION

It has been recognized that the risk of stroke associated with medical procedures can be reduced by using a filter to protect those vessels which are at risk from the procedure.

In view of the need to protect vessels endangered by medical procedures, the present system and method were conceived and developed. In accordance with the system and method, a vessel protector is provided. The protector includes a filtering material arranged in a tube or a tube-like shape when in a filtering configuration, and at least one pull-wire and snare for closing at least one end of the tube or tube-like shape. The protector is collapsible for positioning in a blood vessel, and expandable to the filtering configuration.

### BRIEF DESCRIPTION OF THE DRAWINGS

Various embodiments of the present system and method will now be discussed with reference to the appended drawings. It is to be appreciated that these drawings depict only some embodiments and are therefore not to be considered as limiting the scope of the present system and method.

FIG. 1 is a side perspective view of a vessel protector system in accordance with a first embodiment.

FIG. 2 is a distal end view of the vessel protector of FIG. 1.

FIG. 3 is a side view of the distal end of the vessel protector of FIG. 1 in the closed condition.

FIG. 4 is a highly schematic view showing the vessel protector of FIG. 1 as used in a transcatheter aortic valve implantation (TAVI) procedure.

FIGS. 5A-5C are highly schematic views depicting the recapture of the vessel protector of FIG. 1.

FIG. 6 is a side view of the distal end of a vessel protector according to a second embodiment.

FIG. 7 is a side perspective view of a vessel protector according to a third embodiment.

FIG. 8 is a side view of a vessel protector according to a fourth embodiment.

FIGS. 9A-9C are highly schematic views depicting the closure of the distal end of a vessel protector according to a fifth embodiment.

FIGS. 10A and 10B are side and end perspective views, respectively, of a vessel protector according to a sixth embodiment.

FIG. 11 is a side perspective view of a vessel protector according to a seventh embodiment.

### DETAILED DESCRIPTION

In the description that follows, the terms “proximal” and “distal” are to be taken as relative to a user (e.g., a surgeon or an interventional cardiologist) of the disclosed devices and methods. Accordingly, “proximal” is to be understood as relatively close to the user, and “distal” is to be understood as relatively farther away from the user.

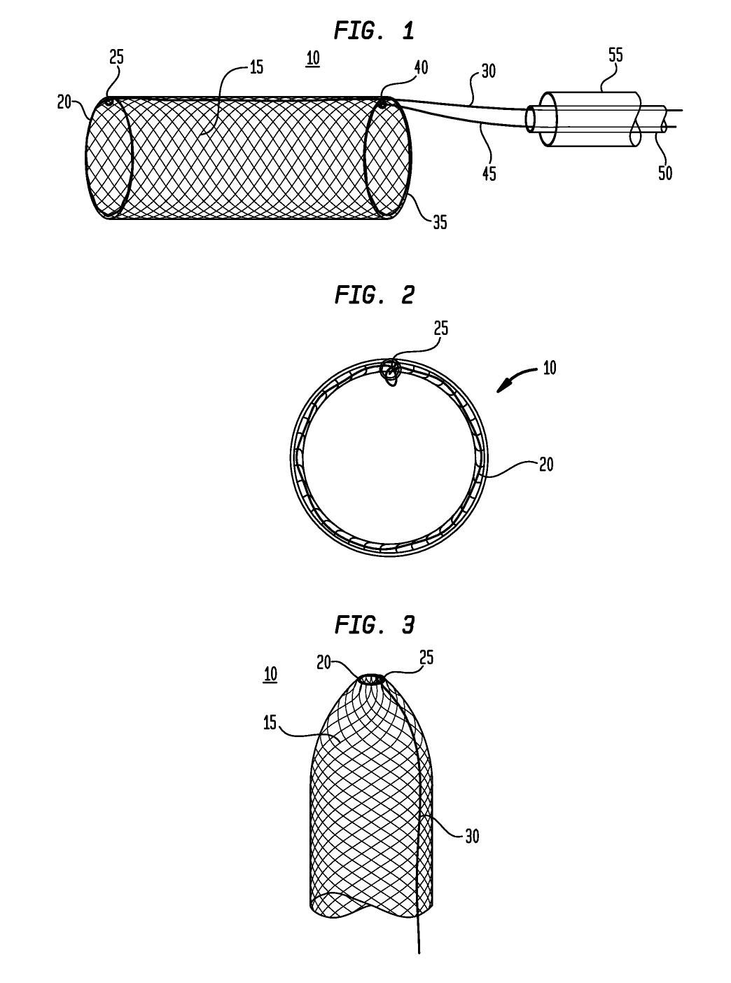

Referring to FIG. 1, there is shown a side perspective view of a vessel protector system in accordance with a first embodiment. As can be seen from FIG. 1, the vessel protector system includes a vessel protector 10. The vessel protector 10 includes a body 15, a distal snare 20 having a slipknot or eyelet 25, a distal pull-wire 30, a proximal snare 35 having a slipknot or eyelet 40, and a proximal pull-wire 45. For additional clarity, the distal snare 20 and slipknot 25 are illustrated in FIG. 2, which depicts the vessel protector 10 of FIG. 1 when viewed from the distal end of the protector.

The body 15 of protector 10 may be formed from a woven, braided, or knitted material having openings of sufficient size to allow the passage of blood, but block the passage of particulates greater than a certain size. As such, the material of body 15 acts as a filter. The body 15 may have a generally hollow cylindrical shape in use, but is collapsible to fit within sheath 55 for delivery into and removal from the patient. In that regard, body 15 may be formed from a shape-memory material, such as nickel titanium alloy (NiTi, or “nitinol”), that is readily collapsible and that will automatically expand to an operative shape upon deployment from sheath 55. For example, body 15 may be formed from braided nitinol wire, from nitinol wire woven to form a mesh, from a nitinol tube perforated with a plurality of small apertures, and other such structures. Alternatively, body 15 may be formed from other metals, metal alloys, or polymers such as polyurethane, nylon or polyethylene, that are capable of being perforated, woven or otherwise formed into a porous hollow cylinder that may be collapsed within sheath 55 for delivery into and removal from the patient, and expanded when deployed from the sheath. In that regard, body 15 may return to its expanded shape automatically when deployed from the sheath, or may be expanded by the application of a radially outward force applied to the body. Still further, body 15 may be formed with a shape-memory or otherwise expandable frame supporting a fabric layer formed from woven or perforated polyester, nylon, polyethylene or similar material. The expansion of the frame will cause the fabric layer to achieve a full cylindrical shape following deployment from sheath 55.

As noted above, the material forming body 15 should have openings of sufficient size to permit the passage of blood, but block the passage of particulates greater than a certain size. In this regard, the openings in body 15 are preferably between about 80 um² and about 300 um².

In some embodiments, the weave, braid, or knit of the protector body 15 or the perforations or other openings therein may be varied such that the size of the openings vary according to their position on the body. For example, a braided protector body may be formed with a varying pick count such that the section generally midway between the ends of the protector body 15 has smaller mesh openings than the sections bordering the ends of the body. Such a protector body can provide finer filtering at its middle area as compared to its end areas. Other variations in opening size along the length of body 15 are also contemplated herein.

Referring back to FIG. 1, the vessel protector system also includes a pull-wire support sleeve 50 and a delivery and retrieval sheath 55. The pull-wires 30 and 45 are slidably disposed within support sleeve 50, and sleeve 50 extends longitudinally through the lumen of sheath 55. The support sleeve 50 is positioned proximate to body 15. In some embodiments, the support sleeve 50 is connected to either a slide-handle or concentric tubes, and is movable relative to sheath 55 and pull-wires 30 and 45 such that the sleeve can advance distally over the pull-wires to the eyelet. The support sleeve 50 may advance to the proximate eyelet 40, or to the distal eyelet 25. Further, the support sleeve 50 may have a coaxial structure, including an outer sleeve and an inner sleeve. In the coaxial embodiment, the outer sleeve may advance to the proximal eyelet 40 while the inner sleeve may advance to the distal eyelet 25.

Sheath 55 may have a diameter sized according to the vessel in which it will be used. For example, when the sheath 55 is to be used in the aorta, the sheath may have a diameter in the range of 8 Fr to 14 Fr, depending on the aortic diameter. When the vessel protector system is being used on a patient, the proximal ends of sheath 55, support sleeve 50, and pull-wires 30 and 45 are located outside the patient's body.

Snares 20 and 35 may be formed of a filamentary material that is interwoven through at least some of the material openings at the distal and proximal ends of body 15, respectively. The material forming snares 20 and 35 should be flexible, yet have sufficient tensile strength to resist breaking when pulled on to close the open ends of body 15. Materials which may be used for snares 20 and 35 include conventional suture material, polymeric filaments, thin metal wires, and other such materials. For example, the materials which may be used for snares 20 and 35 include stainless steel wire and polymer thread.

In one arrangement, a filamentary material may be interwoven through at least some of the material openings at the distal end of body 15, beginning at an entry point on the body and traversing the entire circumference of the body to an exit point adjacent the entry point, so as to form distal snare 20. One tail of the filamentary material may extend as pull-wire 30 proximally along body 15, through support sleeve 50, and out from the proximal end of the support sleeve where it may be grasped by a user. The other tail of the filamentary material may be formed into a slipknot 25 around pull-wire 30.

A similar structure may be formed at the proximal end of body 15. That is, a filamentary material may be interwoven through at least some of the material openings at the proximal end of body 15, beginning at an entry point on the body and traversing the entire circumference of the body to an exit point adjacent the entry point, so as to form proximal snare 35. One tail of the filamentary material may extend as pull-wire 45 proximally from body 15, through support sleeve 50, and out from the proximal end of the support sleeve where it may be grasped by a user. The other tail of the filamentary material may be formed into a slipknot 40 around pull-wire 45. Accordingly, the pull-wires 30 and 45 may be formed from the same materials described above for forming the snares 20 and 35.

It should be noted that, in the FIG. 1 embodiment and the other embodiments described herein, it is not necessary for the pull-wires to be integral with their associated snares and/or slipknots. Rather, the pull-wires may be distinct elements that are connected to their respective snares and, optionally, slipknots. In either case, the pull-wires are to be implemented so that the snares 20 and 35 to which they are coupled close when a pulling force is exerted on the pull-wires. Further, even if the pull-wires are not integral with their associated snares or slipknots, the pull-wires may be formed from conventional suture material, polymeric filaments, thin metal wires, and other such materials, for example, stainless steel wire or polymer thread.

In addition, it should be noted that the term “slipknot” is used to denote actual slipknots, i.e., knots that slide along a thread or other filament or wire around which they are tied, as well as elements that are not actual slipknots but accommodate a sliding thread or other filament or wire. For example, the term “slipknot” may denote an eyelet. Still further, it should be noted that the slipknot opening must be larger than the diameter of the thread, filament or wire forming its associated snare and/or pull-wire in order to enable the snare and/or pull-wire to move through the opening.

FIG. 3 depicts the distal end of the vessel protector of FIG. 1 after it has been closed through operation of pull-wire 30. The proximal end may be similarly closed through use of pull-wire 45. As will be described below, by using pull-wires 30 and 45 to close the ends of body 15, any plaque and/or thrombus captured by the body may be held there as the protector is removed from the patient.

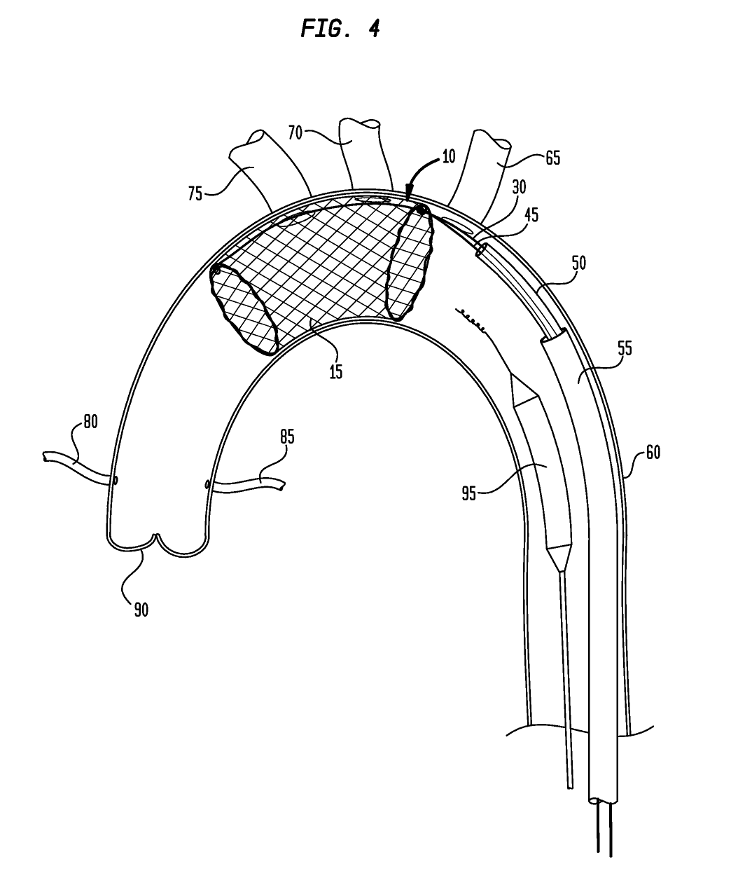

Referring now to FIG. 4, there is shown the vessel protector 10 of FIG. 1 as used in a transcatheter aortic valve implantation (TAVI) procedure. The figure includes representations of a patient's aorta 60, left subclavian artery 65, left common carotid artery 70, brachiocephalic artery 75, right coronary artery 80, left coronary artery 85, and aortic valve 90. A TAVI delivery system 95 has been introduced to the aorta 60 through, for example, a transfemoral approach. The vessel protector 10 has also been introduced to the aorta 60 through, for example, a transfemoral approach. In one implementation, the vessel protector may be deployed within the aorta prior to valve implantation.

The vessel protector 10 is introduced to the aorta 60 in a collapsed configuration within the sheath 55. Sheath is maneuvered along the aorta 60 until it is in proper position for deployment of protector 10. Once sheath 55 is properly positioned, the user pulls the sheath proximally relative to protector 10 to release the body 15. As the sheath continues to be pulled back, release of the support sleeve 50 follows release of the body 15. Once the body 15 has been properly positioned and fully released from the sheath 55, it expands to cover the openings to certain arteries as desired while allowing the TAVI delivery system 95 to pass through its length. That is, the body 15 expands into a tube or tube-like shape that acts to filter blood passing through its wall to an artery, but that is sufficiently large in diameter to permit TAVI delivery system 95 or other instruments to pass through its inner lumen. The protector 10 is held in place by the radial force that the body 15 exerts on the aorta when the body is in the expanded configuration. Accordingly, the diameter of the tubular body 15 in the expanded configuration should be larger than the diameter of the aorta 60 at the position in the aorta at which the body is deployed. More specifically, the diameter of the body 15 in the expanded configuration should be a minimum of 1.05 times the relevant aorta diameter. Nevertheless, the precise ratio of expanded body diameter to aorta diameter depends on the braid/wire size of the body material. By way of example, if the relevant aorta diameter is 25 mm, the diameter of the corresponding body in a fully expanded, unconstrained condition may be about 27 mm.

Body 15 may be self-expanding upon release from sheath 55, or may require the use of one or more instruments to expand following release. A body 15 which is self-expanding may be formed from a biocompatible elastic, superelastic, elastomeric, or shape-memory material which returns to an initial undeformed shape upon release from the sheath 55. Alternatively, a body 15 which is not self-expanding may be formed from a biocompatible material which deforms plastically, and may employ an inflatable balloon or other expandable device to effect radial expansion.

A body 15 formed from a shape-memory material expands as it is released from the sheath 55. That is, the body 15 may begin to expand as it is partially released from the sheath, and may fully expand when fully released from the sheath. For example, a body 15 made of nitinol will fully expand when fully released from the sheath. Alternatively, the body 15 may not fully expand when fully released from the sheath. In any case, the practitioner will have to maneuver the body 15 to the desired position within the vessel.

In any event, in the scenario of FIG. 4, the protector 10 is positioned to protect the left common carotid artery 70 and the brachiocephalic artery 75 from plaque and thrombus that may be released during the valve implantation. That is, the wall of the body 15 covers the openings of the left common carotid and brachiocephalic arteries, with the openings in the filtering material of the body permitting the passage of blood while blocking the passage of plaque and thrombus. Further, while the protector 10 provides filtering, its tubular shape allows for the free passage of instruments through its length.

Upon completion of the valve implantation, instruments used for the implantation may be withdrawn through the body 15 and the protector 10 may be removed from the aorta 60. Prior to removing the protector 10, the ends of body 15 may be cinched closed to trap any plaque or thrombus captured therein. This may be accomplished by grasping the proximal end of pull-wire 30 and pulling proximally thereon while holding body 15 in place to close snare 20 on the distal end of the body. Subsequently, the proximal end of pull-wire 45 may be grasped and pulled proximally while holding body 15 in place to close snare 35 on the proximal end of the body. To provide support for the cinching of the ends of body 15, support sleeve 50 may be advanced distally to slipknot 40 of protector 10. The support sleeve 50 may advance separately from the pull-wires 30 and 45, and thus the pull-wires are arranged to extend out from the proximal end of the support sleeve. Some embodiments include a proximal support sleeve and a distal support sleeve, the proximal support sleeve advancing to the proximal eyelet 40 or the proximal end of body 15, and the distal support sleeve advancing to the distal eyelet 25 or the distal end of body 15. In other embodiments, only a distal support sleeve is included, with the delivery sheath 55 serving as a proximal support sleeve.

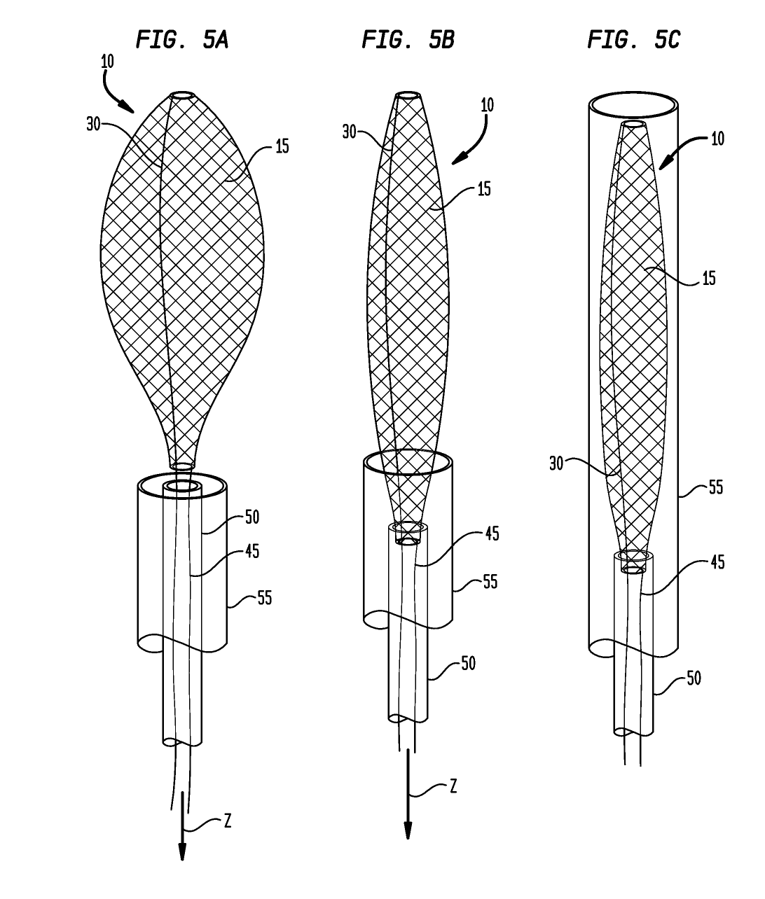

With both of snares 20 and 35 closed, as shown in FIG. 5A, any plaque or thrombus captured in body 15 may be held therein during removal of protector 10 from the patient. It will be appreciated, of course, that the order in which the ends of body 15 are closed is not critical—either distal end first or proximal end first, although typically the upstream end, in this case the distal end, would be closed first in order to prevent debris from escaping at a point where it could flow into a protected vessel. Once both ends of body 15 have been closed, body 15 may be retracted into sheath 55 for removal.

As can be seen in FIG. 3, the actuation of a snare to close an end of body 15 causes the end of the body to become tapered. This taper enables the proximal end of body 15 to be received in the distal end of sheath 55 as the proximal pull-wire 45 and sleeve 50 are pulled in the direction of arrow Z, as shown in FIG. 5A. Body 15 gradually collapses and elongates as it is pulled into sheath 55, as shown in FIG. 5B. (It is noted that a similar state exists upon deployment of the protector 10, and that the figure is therefore illustrative of the foreshortening of the protector that occurs upon deployment.) FIG. 5C shows body 15 in its fully recaptured state. As can be seen from the figure, body 15 is elongated and conforms to the shape of sheath 55. Once fully retracted into sheath 55, protector 10 may be removed from the patient. Any captured material will be removed from the patient along with protector 10, and thus will not present a threat of embolism.

It should be noted that FIG. 4 depicts an illustrative application of protector 10, and that applications of the protector are not limited to the context of FIG. 4. For example, protector 10 may be delivered to the patient's aorta 60 through the left radial artery, left brachial artery, or left subclavian artery. Moreover, protector 10 may be used to protect vessels other than the left common carotid and brachiocephalic arteries, and may be employed in procedures other than TAVI procedures. Thus, protector 10 may be used in any procedure in which there is a possibility that plaque, thrombus or other debris may be introduced into the bloodstream, and in which the protector may be positioned to capture same.

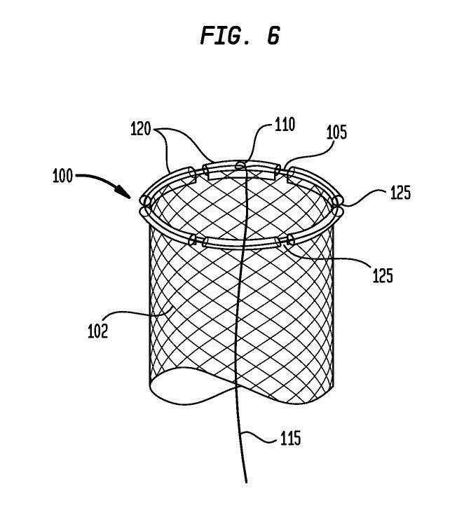

Referring now to FIG. 6, there is shown a vessel protector 100 in accordance with a second embodiment. The protector 100 includes a protector body 102, a distal snare 105 having a slipknot 110, and a distal pull-wire 115, all of which may be substantially the same as the body, distal snare, and slipknot described above in connection with protector 10. Protector 100 may also include one more wraps 120 disposed on the distal end of body 102. The wraps 120 may be folded over the distal end of body 102. Also, the wraps 120 may be integral with a liner material which is included as part of the body 102. For example, a liner with integral wraps may be made of polyurethane, another elastomeric polymer, and/or a fabric such as nylon. The liner with integral wraps may then be used with a braided body, for instance. Further, the wraps 120 may be provided as one layer of material on the outer surface of the body 102, one layer of material on the inner surface of the body, or as two layers of material which sandwich the body.

The wraps 120 serve to ease movement of the snare 105 such that, when pull-wire 115 is pulled, the snare readily closes with reduced contact between the snare and the body 102 of the protector. Openings 125 may be provided between adjacent segments of the wraps to enable the distal end of body 102 to collapse readily during cinching of snare 105 in order to realize tighter closure. The openings 125 also reduce the bulk of the protector 100 so that the protector can collapse to a smaller size for fitting within a delivery/removal sheath, such as sheath 55 described above.

Although not shown in FIG. 6, protector 100 may also include a proximal snare and proximal pull-wire for the purpose of cinching the proximal end of the protector. The proximal end of the protector 100 may have a structure similar to the proximal end of protector 10 described above. Alternatively, the proximal end of protector 100 may have one or more wraps 120 to facilitate the closure of the proximal end upon activation of the proximal pull-wire.

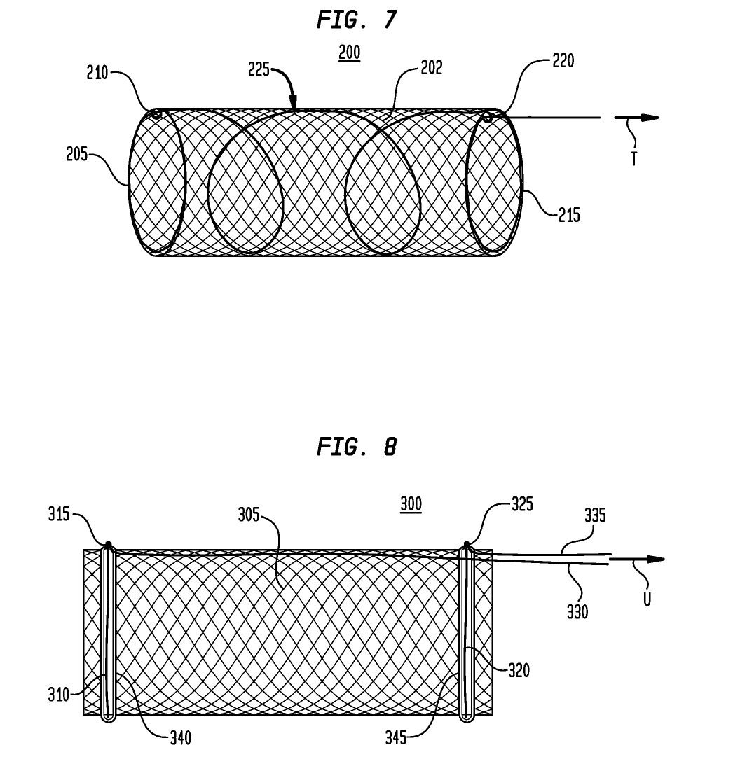

Referring now to FIG. 7, there is shown a side perspective view of a vessel protector 200 in accordance with a third embodiment. Protector 200 includes a mesh body 202 that is not self-supporting. That is, body 202 is not made of a self-expanding shape-memory material or an expandable plastic material. In order to provide for expansion of body 202 upon release from a sheath, such as sheath 55 of FIG. 1, protector 200 includes a pull-wire 225 that spirals through body 202. Pull-wire 225 may be formed from an elastic, superelastic, or elastomeric material, including a shape-memory material, and may be connected to a distal snare 205 having a slipknot 210, and a proximal snare 215 having a slipknot 220, in a manner similar to that described above in connection with protector 10 of FIG. 1. Further, the pull-wire 225 may be integral with one or both of snares 205 and 215. Upon deployment of protector 200 from sheath 55, the pull-wire 225 will automatically expand radially, drawing body 202 into the open expanded shape shown in FIG. 7.

To entrap any debris captured by body 202, a pulling force may be applied to pull-wire 225 in the direction of arrow T in FIG. 7. When such pulling force is applied to pull-wire 225, both snares 205 and 215 of protector 200 close to entrap any debris within body 202. Furthermore, pull-wire 225 will radially collapse when pulled to form small diameter spirals, drawing the remainder of body 202 into a radially collapsed configuration. Protector 200 may then be removed from the patient in a manner similar to that described in connection with FIGS. 4 and 5A-5C.

FIG. 8 is a side view of a vessel protector 300 in accordance with a fourth embodiment. The vessel protector 300 includes a braided nitinol body 305, a distal snare 310 having a slipknot 315, a proximal snare 320 having a slipknot 325, a distal pull-wire 330, and a proximal pull-wire 335. Protector 300 may have substantially the same structure as protector 10 of FIG. 1, with the exception that protector 300 includes a tubular distal band 340 and a tubular proximal band 345. Distal snare 310 may be positioned within distal band 340, and proximal snare 320 may be positioned within proximal band 345. Distal band 340 and proximal band 345 may be formed from a fabric, a polymer film, such as a polyurethane film, or other elastomeric material which is sewn or otherwise attached to the braided material of body 305 on the outer surface of the body. The material of bands 340 and 345 keeps the snares 310 and 320, respectively, in place and facilitates the cinching of the snares without binding. That is, when a pulling force is exerted on distal pull-wire 330 in the direction of arrow U in FIG. 8, distal snare 310 closes in a cinching manner and distal band 340 closes along with the distal snare. Similarly, when a pulling force is exerted on proximal pull-wire 335 in the direction of arrow U, proximal snare 320 closes in a cinching manner and proximal band 345 closes along with the proximal snare. Bands 340 and 345 may or may not shorten from a stretched condition as snares 310 and 320 close. In any event, when pull-wires 330 and 335 are pulled, the bands deform from their tubular shape so that the bands can be retracted into a delivery sheath.

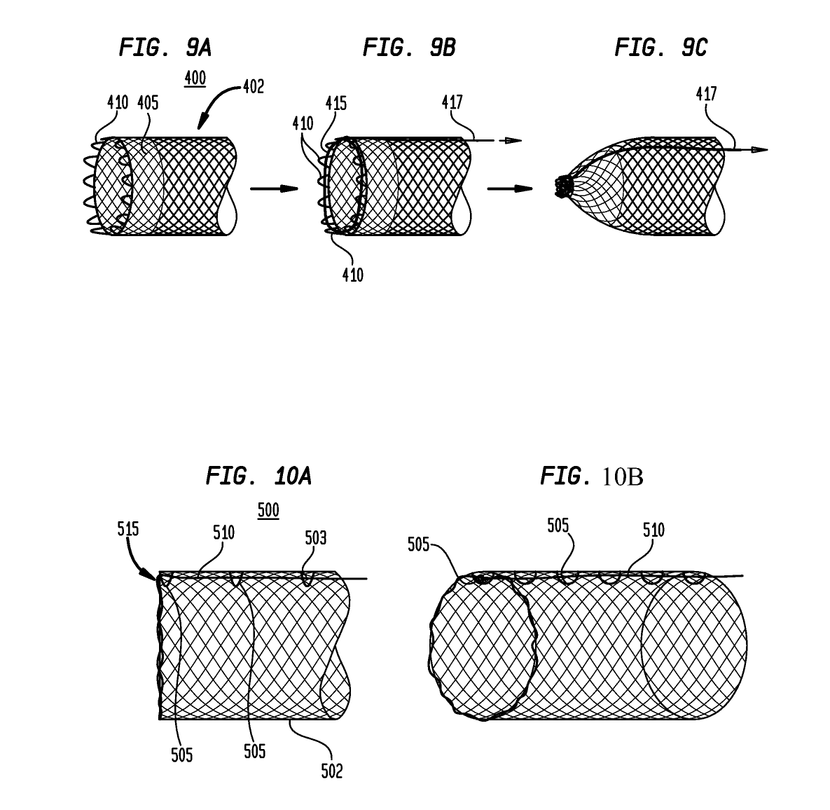

FIGS. 9A-9C depict a vessel protector 400 in accordance with a fifth embodiment. Protector 400 includes a protector body 402 having a mesh structure made of braided or woven nitinol. As can be seen in FIG. 9A, protector 400 includes a section 405 (or “folded portion”) in which body 402 has been folded back upon itself. Along the folded edge of section 405, loops 410 are picked out from the braided or woven material so as to protrude from the distal end of body 402. Folded portion 405 and loops 410 may be fixed in place by, for example, heat-setting. In this manner, loops 410 provide a channel for a distal snare. FIG. 9B shows how a distal snare 415 may be passed through loops 410. Snare 415 is coupled to a distal pull-wire 417, and FIG. 9C shows how snare 415 and pull-wire 417 may be used to cinch the distal end of body 402. Although, not shown in FIGS. 9A-9C, a configuration of loops, a snare and a pull-wire similar to those employed at the distal end of body 402 may be employed at the proximal end of the body for purposes of cinching the proximal end of the body.

FIGS. 10A and 10B are side and perspective views, respectively, of a vessel protector 500 in accordance with a sixth embodiment. Protector 500 includes a body 502, which may be a mesh structure made of braided or woven nitinol, and a channel 503 provided along the longitudinal direction of the body. Channel 503 may be formed by picking out loops 505 from the braided or woven material of body 502 and orienting them radially inward, toward the longitudinal axis of the body. Once the loops have been oriented, they may be heat-set in place, thereby forming channel 503 longitudinally along the luminal surface of body 502. Channel 503 may be used to guide a pull-wire 510 along the length of body 502 from a distal snare 515 to the proximal end of body 502, such that when a pulling force is exerted on the wire the snare cinches closed the distal end of the body. As the distal end is cinched closed, channel 503 provides support for pull-wire 510 to insure that the wire acts to close snare 515 rather than bend body 502. Although not shown in FIGS. 10A and 10B, protector 500 may also include a proximal snare and a proximal pull-wire in a manner similar to protector 10 in FIG. 1.

In an alternative to the embodiment of FIGS. 10A and 10B, loops 505 may be oriented radially outward, away from the longitudinal axis of body 502, to form a channel along the abluminal surface of the body. Optionally, protector 500 may include a fabric sleeve for providing added support for pull-wire 510. For example, a sleeve may be inserted into channel 503 provided by loops 505 of FIG. 10A, whether on the luminal or abluminal surface of body 502, and pull-wire 510 may be inserted into the sleeve.

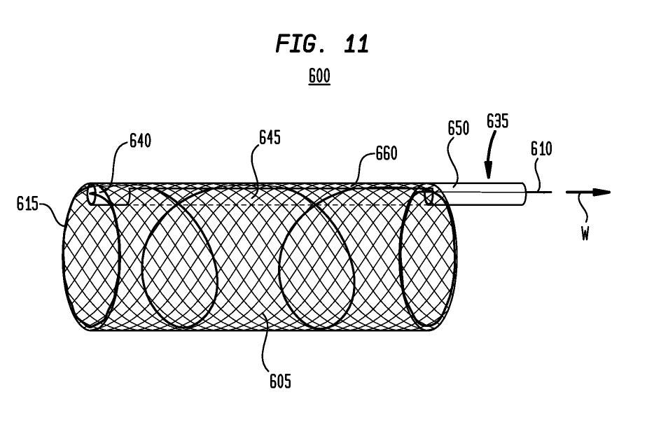

Referring now to FIG. 11, a vessel protector 600 in accordance with a seventh embodiment is shown. Protector 600 is substantially the same as protector 200 of FIG. 7, with the exception that protector 600 includes a support sleeve 635. Support sleeve 635 may be formed from a continuous hypo-tube having a section removed from its middle portion so as to define a tubular distal portion 640, a tubular proximal portion 650, and an elongated opening 645 located substantially toward the middle of the tube and facing toward the longitudinal axis of the protector body 605. The remainder of the hypo-tube between the distal portion 640 and the proximal portion 650 defines spine 660 which may support the distal and proximal portions and prevents the fabric of body 605 from collapsing upon itself longitudinally during retraction of the body into a delivery/removal sheath. Sleeve 635 supports body 605 to insure that when a pulling force is applied to pull-wire 610 in the direction of arrow W, the pull-wire acts to close snare 615 rather than merely bend the body. The sleeve 635 also acts as a support for facilitating cinching of the proximal end of the protector 600 and retraction of the protector. Distal portion 640 and proximal portion 650 of sleeve 635 provide channels to guide pull-wire 610, spine 660 supports pull-wire 610 to the distal end of protector 600, and elongated opening 645 enables pull-wire 610 to achieve the expanded configuration during deployment of protector 600, and to contract and pass into the sleeve during recapture of the protector.

The protector 600 may be delivered and retrieved using a sheath that is the same as or similar to sheath 55 of FIG. 1. For delivery, the body 605 and pull-wire 610 of protector 600 are initially collapsed within the delivery/retrieval sheath. The pull-wire 610 expands as it is released from the sheath in response to a practitioner pushing the support sleeve 635 from the sleeve's proximal end. As the pull-wire 610 is released, it changes from its collapsed shape, which may be that of a straight or approximately straight wire, into its expanded shape, which may be a spiral shape. As pull-wire 610 expands, it draws body 605 into the expanded configuration. To retrieve the protector 600, the practitioner pulls on the support sleeve 635 from the sleeve's proximal end, thereby forcing the body 605 and pull-wire 610 back into the constraints of the sheath, where they are forced to compress back into their collapsed shape.

In some embodiments, the protector 600 of FIG. 11 includes a second snare and a second pull-wire. In such embodiments, the second snare is positioned at the proximal end of the protector 600, and the second pull-wire is coupled to the second snare and acts to close the second snare when pulled in direction W. When the second pull-wire is pulled to close the second snare, sleeve 635 acts as a support for facilitating cinching of the proximal end of protector 600 and retraction of the protector.

Yet another embodiment of a vessel protector may be substantially similar to the vessel protector 10 of FIG. 1. However, rather than having a body formed from a self-supporting material that automatically expands when deployed, the body may be formed from a tube of a braided or woven fabric or other porous material (such as, for example, laser cut polyurethane or expanded polytetrafluorethylene) that is not self-supporting. The tube of fabric may be supported by a shape-memory frame. For example, the body may include a wire of a shape-memory material which extends in the form of a spiral along the length of the body. The frame may be compressed to a collapsed or straightened configuration so as to be loaded into a delivery/retrieval sheath, but upon deployment of the protector may automatically expand radially, drawing the tube of material to an open expanded shape, and supporting the tube in such expanded shape. The frame may again be readily compressed upon recapture of the body within the sheath. Moreover, the distal and proximal ends of the frame may be readily collapsed upon actuation of one or more pull-wires to close the ends of the body, as described above in connection with vessel protector 10. Thus, the shape-memory frame may perform some of the same functions as pull-wire 225 of FIG. 7 and pull-wire 610 of FIG. 11. However, the shape-memory frame is not itself a pull-wire nor part of a pull-wire. Rather, the shape-memory frame may be part of a vessel protector that includes one or more pull-wires that are distinct from the frame.

Although the system and method herein have been described with reference to particular embodiments, it is to be understood that these embodiments are merely illustrative of the principles and applications of the present system and method. It is therefore to be understood that numerous modifications may be made to the illustrative embodiments and that other arrangements may be devised without departing from the spirit and scope of the present system and method as defined by the appended claims.

It will be appreciated that the various dependent claims and the features set forth therein can be combined in different ways than presented in the initial claims. It will also be appreciated that the features described in connection with individual embodiments may be shared with others of the described embodiments.

1. A vessel protector, comprising:
a body formed from a filtering material, the body having a collapsed configuration and a tubular expanded configuration with an open proximal end and an open distal end;
a first snare attached to one end of the body; and
a first pull-wire connected to the first snare, whereby the exertion of a pulling force on the first pull-wire contracts the first snare and thereby closes the one end of the body.

2. The vessel protector according to claim 1, wherein the filtering material is a mesh.

3. The vessel protector according to claim 1, wherein the filtering material is a braided material.

4. The vessel protector according to claim 1, wherein the filtering material is formed from a shape memory material.

5. The vessel protector according to claim 1, wherein the first pull-wire is formed integrally with the first snare.

6. The vessel protector according to claim 1, wherein the first pull-wire is formed separately from the first snare.

7. The vessel protector according to claim 1, wherein the first snare is at least partially enclosed by one or more wraps.

8. The vessel protector according to claim 1, wherein the filtering material is self-supporting.

9. The vessel protector according to claim 1, wherein the filtering material is not self-supporting, and the body includes a self-supporting frame for supporting the filtering material.

10. The vessel protector according to claim 9, wherein the first pull-wire includes the self-supporting frame.

11. The vessel protector according to claim 1, further comprising a multiplicity of loops on the one end of the body that collectively define a channel for the first snare.

12. The vessel protector according to claim 1, further comprising a multiplicity of loops along a length of the body that collectively define a channel for the first pull-wire.

13. The vessel protector according to claim 1, further comprising:
a second snare attached to another end of the body; and
a second pull-wire connected to the second snare,
whereby exertion of a pulling force on the second pull-wire contracts the second snare and thereby closes the another end of the body.

14. The vessel protector according to claim 1, further comprising a hypo-tube extending from the distal end of the body to the proximal end of the body, the first pull-wire extending through the hypo-tube.

15. The vessel protector according to claim 18, wherein the hypo-tube has a distal portion, a proximal portion, and an elongated opening between the distal portion and the proximal portion, and the first pull-wire has a collapsed condition and a radially expanded condition, the first pull-wire projecting out from the elongated opening of the hypo-tube in the radially expanded condition.

16. A vessel protector system, comprising:
a vessel protector having a body formed from a filtering material, the body having a collapsed configuration and a tubular expanded configuration with an open proximal end and an open distal end; a snare attached to one end of the body; and a pull-wire connected to the snare, whereby exertion of a pulling force on the pull-wire contracts the snare and thereby closes the one end of the body; and
a sheath movable relative to the body between a first position in which the sheath covers the body and holds the body in the collapsed configuration, and a second position in which the sheath uncovers the body and releases the body for movement to the expanded configuration.

17. The vessel protector system according to claim 16, further comprising a sleeve through which the pull-wire passes, the sleeve being positioned within the sheath when the body is in the collapsed configuration and being positioned outside of the sheath when the body is in the expanded configuration.

18. A method for protecting blood vessels during a medical procedure, comprising:
inserting a vessel protector system into a patient's body, the vessel protector system including a vessel protector and a sheath, the vessel protector having a body formed from a filtering material and having a collapsed configuration and a tubular expanded configuration, the sheath being movable relative to the body between a first position for holding the body in the collapsed configuration, and a second position for releasing the body for movement to the expanded configuration;
positioning the vessel protector system adjacent an open end of at least one blood vessel; and
moving the sheath to the second position to deploy the body, whereby the body moves to the tubular expanded configuration covering the open end of the at least one blood vessel to filter blood passing through the body into the at least one blood vessel.

19. The method according to claim 18, further comprising:
closing open ends of the body in the tubular expanded configuration;
moving the sheath to the first position to compress the body to the collapsed configuration; and
removing the vessel protector system from the patient's body.
